(19) United States
(12) Patent Application Publication
(10) Pub. No.: US 2018/0353736 A1
(43) Pub. Date: Dec. 13, 2018

(54) BIMODAL SHUNT

(71) Applicant: Thomas O. Viker, New Brighton, MN (US)

(72) Inventor: Thomas O. Viker, New Brighton, MN (US)

(21) Appl. No.: 16/003,831

(22) Filed: Jun. 8, 2018

Related U.S. Application Data
(60) Provisional application No. 62/517,676, filed on Jun. 9, 2017.

(51) Int. Cl. A61M 27/00; A61M 39/24

(52) U.S. Cl. CPC A61M 27/006; A61M 2025/0078; A61M 39/24

(57) ABSTRACT

A shunt can include a primary catheter for receiving fluid, a secondary catheter for receiving fluid, and a pressure-sensitive valve to prevent flow of fluid to the secondary catheter in a first mode until the primary catheter is occluded. The pressure-sensitive valve can transition to a second mode responsive to occlusion of the primary catheter, permitting flow of fluid to the secondary catheter.

### CROSS-REFERENCE TO RELATED APPLICATIONS

This application claims the benefit of the filing date of U.S. Provisional Application Ser. No. 62/517,676, filed on Jun. 9, 2017, the disclosure of which is incorporated by reference herein.

### BACKGROUND

Shunts can be used to drain fluid. For example, cerebral spinal fluid shunts can be used to treat hydrocephalus by draining excess fluid from the brain to another part of the body where the fluid can be absorbed as part of the circulatory process. If the shunt fails due to occlusion or otherwise, it must be replaced or flushed to provide proper draining. Such failures can be harmful, labor intensive, time consuming, costly, or the like.

### OVERVIEW

To better illustrate the instrument disclosed herein, a non-limiting list of examples is provided here:

In Example 1, a shunt can include a primary catheter configured to receive cerebral spinal fluid, a secondary catheter configured to receive cerebral spinal fluid, and a first pressure-sensitive valve positioned to prevent flow of cerebral spinal fluid to the secondary catheter in a first mode, the first pressure-sensitive valve configured to transition to a second mode responsive to occlusion of the primary catheter, permitting flow of cerebral spinal fluid to the secondary catheter.

In Example 2, the shunt of Example 1 is optionally configured such that the primary and secondary catheters are ventricular catheters.

In Example 3, the shunt of Example 2 is optionally configured such that the pressure-sensitive valve is positioned at a proximal end of the secondary catheter, and the pressure-sensitive valve is configured to transition to the second mode responsive to a threshold intracranial pressure.

In Example 4, the shunt of Example 1 is optionally configured such that the first pressure-sensitive valve can include a ball and at least one wall feature, the at least one wall feature configured to restrict movement of the ball.

In Example 5, the shunt of Example 1, can optionally include a second pressure-sensitive valve positioned at a distal end of the secondary catheter to block flow through the distal end of the secondary catheter in a first mode and configured to transition to a second mode responsive to a threshold pressure in the secondary catheter.

In Example 6, the shunt of Example 1, can optionally include a chamber coupled to a proximal end of the secondary catheter that is optionally configured such that the first pressure-sensitive valve is configured to transition to the second mode responsive to a threshold pressure in the chamber.

In Example 7, a shunt can include a chamber body configured to receive fluid, a primary distal catheter coupled at a proximal end to the chamber body and configured to fluid from the chamber body, a secondary distal catheter coupled at a proximal end to the chamber body and configured to receive fluid from the chamber body, and a first pressure-sensitive valve configured to prevent flow to the secondary distal catheter from the chamber body in a first mode until the primary distal catheter is occluded such that a threshold pressure is reached in the chamber body to transition the first pressure-sensitive valve to a second mode.

In Example 8, the shunt of Example 7 is optionally configured such that the chamber body can include a primary chamber and a secondary chamber, and the first pressure-sensitive valve is positioned to prevent flow to the secondary chamber until a threshold pressure is reached in the primary chamber.

In Example 9, the shunt of Example 7, can optionally include a pressure modulation valve configured to modulate the flow of cerebral spinal fluid to the chamber body.

In Example 10, the shunt of Example 7, can optionally include a second pressure-sensitive valve positioned at a distal end of the secondary distal catheter to prevent flow through the distal end of the secondary distal catheter in a first mode, the second pressure-sensitive valve configured to transition to a second mode responsive to a threshold pressure in the secondary distal catheter.

In Example 11, the shunt of Example 7 is optionally configured such that the primary and secondary distal catheters are coupled together along a portion of their lengths.

In Example 12, the shunt of Example 7 is optionally configured such that the first pressure-sensitive valve can include a fusible link.

In Example 13, the shunt of Example 7 is optionally configured such that a distal end of the secondary distal catheter is configured to be positioned away from a distal end of the primary distal catheter, such that the distal ends of the primary and secondary distal catheters can be splayed apart.

In Example 14, the shunt of Example 13 is optionally configured such that the distal ends of the primary and secondary distal catheters are configured to be positioned together.

In Example 15, a shunt can include a proximal catheter configured to receive fluid for draining, a chamber body configured to receive fluid from the proximal catheter, the chamber body including a primary chamber and a secondary chamber, and a first pressure-sensitive valve positioned in the chamber body to prevent flow of fluid to the secondary chamber in a first mode and configured to transition to a second mode responsive to a threshold pressure in the primary chamber.

In Example 16, the shunt of Example 15, can optionally include a primary distal catheter in fluid communication with the primary chamber, and a secondary distal catheter in fluid communication with the secondary chamber, the shunt is optionally configured such that the first pressure-sensitive valve is configured to permit flow to the secondary distal catheter responsive to failure of the primary distal catheter.

In Example 17, the shunt of Example 16, can optionally include a second pressure-sensitive valve positioned at a distal end of the secondary distal catheter and configured to block flow through the distal end of the secondary distal catheter in a first mode, and transition to a second mode responsive to a threshold pressure in the secondary distal catheter.

In Example 18, the shunt of Example 17 is optionally configured such that the second pressure-sensitive valve can include a friction fit ball.

In Example 19, the shunt of Example 15 is optionally configured such that the first pressure-sensitive valve can include a ball and at least one wall feature configured to restrict movement of the ball.

In Example 20, the shunt of Example 15, can optionally include a pressure modulation valve coupled to a distal end of the proximal catheter and configured to receive fluid from the proximal catheter.

In Example 21, a method can optionally include providing an implantable chamber body, forming a proximal catheter port on the chamber body, arranging a pressure-sensitive valve in the chamber body, the valve including a first mode and a second mode, forming a primary distal catheter port on the chamber body, and forming a secondary distal catheter port on the chamber body, wherein the pressure-sensitive valve permits the primary distal catheter port to be in fluid communication with the proximal catheter port and blocks the secondary distal catheter port from being in fluid communication with the proximal catheter in the first mode, and wherein the pressure-sensitive valve permits the distal catheter port to be in fluid communication with the proximal catheter port in the second mode.

In Example 22, the method of Example 21 is optionally configured such that arranging the pressure-sensitive valve in the chamber body includes affixing a ball in a seat.

In Example 23, the method of Example 21 is optionally configured such that arranging the pressure-sensitive valve in the chamber body includes providing one or more wall features to maintain patency of the secondary distal catheter port when the pressure-sensitive valve is in the second mode.

In Example 24, the method of Example 23 is optionally configured such that arranging the pressure-sensitive valve in the chamber body includes configuring the pressure-sensitive valve to transition from the first mode to the second mode responsive to a threshold pressure in the chamber body.

In Example 25, the method of Example 21 is optionally configured such that arranging the pressure-sensitive valve in the chamber body includes configuring the pressure-sensitive valve to irrevocably remain in the second mode.

In Example 26, the method of Example 21 can optionally include coupling a proximal catheter to the proximal catheter port, coupling a primary distal catheter to the primary distal catheter port, and coupling a secondary distal catheter to the secondary distal catheter port.

In Example 27, the method of Example 26 can optionally include arranging a distal valve at a distal end of the secondary distal catheter.

In Example 28, the method of Example 27 is optionally configured such that the distal valve includes a pressure-sensitive valve including a first mode restricting fluid flow through the distal end of the secondary distal catheter and a second mode permitting fluid flow through the distal end of the secondary distal catheter.

In Example 29, the method of Example 28 is optionally configured such that the pressure-sensitive valve is configured to transition from the first mode to the second mode responsive to a threshold fluid pressure in the secondary distal catheter.

In Example 30, the method of Example 29 is optionally configured such that the distal valve plugs the distal end of the secondary distal catheter in the first mode and is configured to eject from the distal end in the second mode.

In Example 31, the system or method of any of Examples 1-30 can optionally be combined.

These and other examples and features of the present devices, systems, and methods will be set forth in part in the following Detailed Description. This overview is intended to provide a summary of subject matter of the present patent application. It is not intended to provide an exclusive or exhaustive removal of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagram of a patient having an implanted bimodal cerebral spinal fluid shunt, in accordance with at least one example of the present disclosure.

FIG. 2A is a view of a portion of a bimodal cerebral spinal fluid shunt, in accordance with at least one example of the present disclosure.

FIG. 2B is a cross-section view of the bimodal cerebral spinal fluid shunt of FIG. 2A taken at cut line 2B-2B, in accordance with at least one example of the present disclosure.

FIG. 3A is a view of a portion of a bimodal cerebral spinal fluid shunt, in accordance with at least one example of the present disclosure.

FIG. 3B is a cross-section view of the bimodal cerebral spinal fluid shunt of FIG. 3A taken at cut line 3B-3B, in accordance with at least one example of the present disclosure.

FIG. 4 is a diagram of a patient having an implanted bimodal cerebral spinal fluid shunt, in accordance with at least one example of the present disclosure.

FIG. 5A is view of a portion of a bimodal cerebral spinal fluid shunt, in accordance with at least one example of the present disclosure.

FIG. 5B is a cross-section view of the bimodal cerebral spinal fluid shunt of FIG. 5A taken at cut line 5B-5B, in accordance with at least one example of the present disclosure.

FIG. 6 is a flow chart illustrating a method, in accordance with at least one example of the present disclosure.

### DETAILED DESCRIPTION

Current cerebral spinal fluid shunts can fail, for example, when the catheter is occluded by a pseudocyst growing over an end of the catheter. When the catheter is occluded, the shunt can no longer effectively drain the cerebral spinal fluid, which typically requires surgery to replace the shunt. The present disclosure provides a bimodal cerebral spinal fluid shunt that increases the average time to system failure by providing at least two different pathways for flow of cerebral spinal fluid. For the purpose of this disclosure, a bimodal shunt includes at least two different pathways for drainage through the shunt. For example, the shunt can have more than one proximal passage, more than one distal passage, or a combination of these.

Modular redundancy can be achieved passively by putting multiple instances of an element into the system, in which the exposure to risk of the elements begins at the same time. Double modular redundancy increases the time to system failure over a non-redundant system by the standard deviation times the square root of two. A greater increase in time to failure can be achieved by an active system in which the redundant element is maintained in pristine condition (e.g., sealed at both ends such that its lumen does not receive the patient's anatomy) and only comes into use when the original fails. For double redundancy systems, the time to failure doubles. In at least one example, the present disclosure provides a bimodal cerebral spinal fluid shunt in which a secondary catheter is maintained in pristine condition until the primary catheter fails, at which point flow is automatically diverted to the secondary catheter.

While the disclosure describes the bimodal shunt with reference to a hydrocephalus cerebral spinal fluid shunt, in at least one example, a bimodal shunt or catheter can be provided for purposes other than cerebral spinal fluid draining. Further, while the disclosure describes a bimodal shunt or a system having double redundancy, in some examples, the system can have more than two modes, or more than double redundancy, for example triple or quadruple redundancy. Additionally, while the disclosure is described with reference to modular redundancy, in some examples the redundancy could be non-modular, such that the different modes are not achieved by identical elements. While the modes of the shunt are described with reference to a first closed mode and a second opened mode, in some examples these can be reversed, the first and second modes could represent different functions, there could be more than two modes, a combination of these, or the like. The applications and possible variations of such examples will be understood by one of ordinary skill in the art.

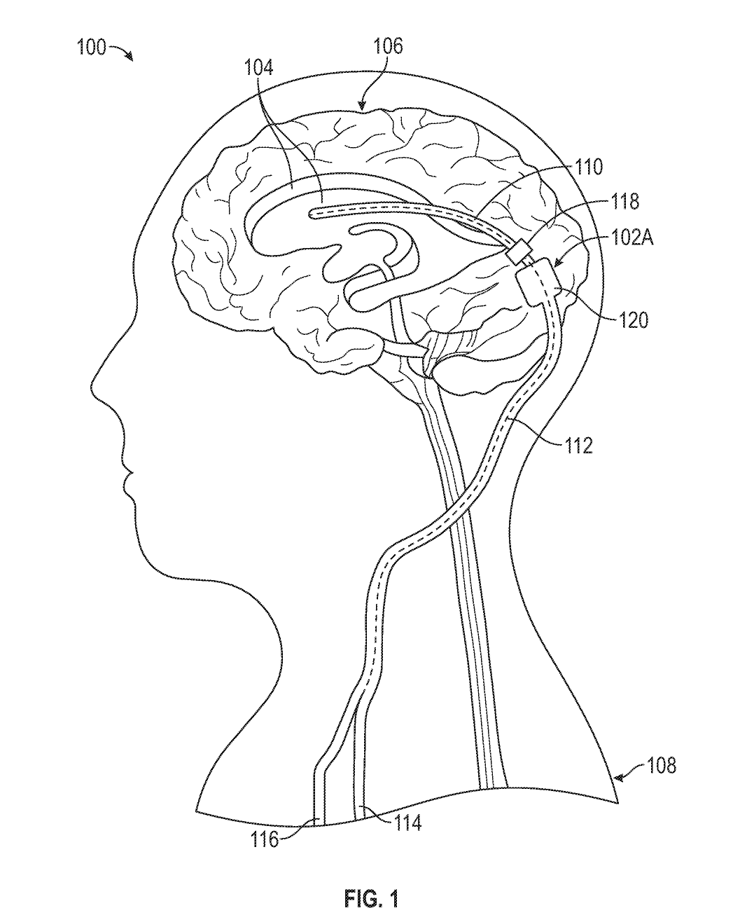

FIG. 1 is a diagram of a patient 100 having an implanted bimodal cerebral spinal fluid shunt 102A (“bimodal shunt”), in accordance with at least one example of the present disclosure. In the illustrated example, the bimodal shunt 102A is shown implanted such that it can drain spinal fluid from the ventricles 104 of the brain 106 and into the torso 108 (e.g., the chest or abdomen) of the patient 100. In some examples, the bimodal shunt 102A can include a proximal catheter 110, and a distal catheter 112. Generally, the bimodal shunt 102A drains fluid; in the case of the illustrated example, the bimodal shunt 102A drains cerebral spinal fluid from the brain 106 of the patient into a proximal end of the proximal catheter 110 and through a distal end of the distal catheter 112 to the patient's torso 108. In at least one example, the proximal catheter 110 is a ventricular catheter. In at least one example, the distal catheter 112 can include redundancy or otherwise be bimodal, such that the distal catheter 112 can include a primary distal catheter 114 and a secondary distal catheter 116. In some examples, the bimodal shunt 102A can include a pressure modulation valve 118 to control the flow of spinal fluid to the distal catheter 112. In some examples, the bimodal shunt 102A can include a chamber body 120 to control flow of spinal fluid to the secondary distal catheter 116.

In the illustrated example, the proximal catheter 110 can drain spinal fluid from the ventricles 104, which can pass through the pressure modulation valve 118 through the chamber body 120 and into the distal catheter 112, where the spinal fluid can be drained into a portion of the torso of the patient 100 to be absorbed into the body. Initially, the spinal fluid can continue from the chamber body 120 to the primary distal catheter 114, until the primary distal catheter 114 fails. For example, if the primary distal catheter 114 becomes occluded, the spinal fluid will accumulate in the primary distal catheter 114 and the chamber body 120 until a threshold pressure is reached in the chamber body 120. The threshold pressure can be any predetermined amount of pressure that indicates that the primary distal catheter 114 is not properly distributing the spinal fluid. In at least one example, the threshold pressure in the chamber body 120 can be indicative of an increased intracranial pressure. For example, in at least one example, the threshold pressure can be indicative of at least 20 cm water intracranial pressure, 15 mmHg intracranial pressure, significantly above normal intracranial pressure, or the like. In at least one example, the threshold pressure can be about 15 mmHg. In some examples, when the threshold pressure is exceeded, the fluid is permitted to flow from the chamber body 120 to the secondary distal catheter 116.

When the occluded primary distal catheter 114 causes the threshold pressure to be reached in the chamber body 120, fluid flows from the chamber body 120 to the secondary distal catheter 116. That is, in at least one example, only one of the primary distal catheter 114 and the secondary distal catheter 116 is used at a time. In at least one example, while the primary distal catheter 114 is active, the secondary distal catheter 116 is blocked off at both ends to maintain a pristine (e.g., dry) catheter. Maintaining a pristine or dry catheter is important to extending the life of the system, since it avoids exposure to causes of occlusion. In at least one example, when the primary distal catheter 114 is occluded, it can be abandoned and flow can be directed to the secondary distal catheter 116. In at least one example, the bimodal shunt 102A can continue draining fluid even after the primary distal catheter 114 has been occluded, such that the bimodal shunt 102A need not be replaced upon occlusion of the primary distal catheter 114, extending the life of the cerebral spinal shunt and avoiding an additional operation and any associated risk and cost.

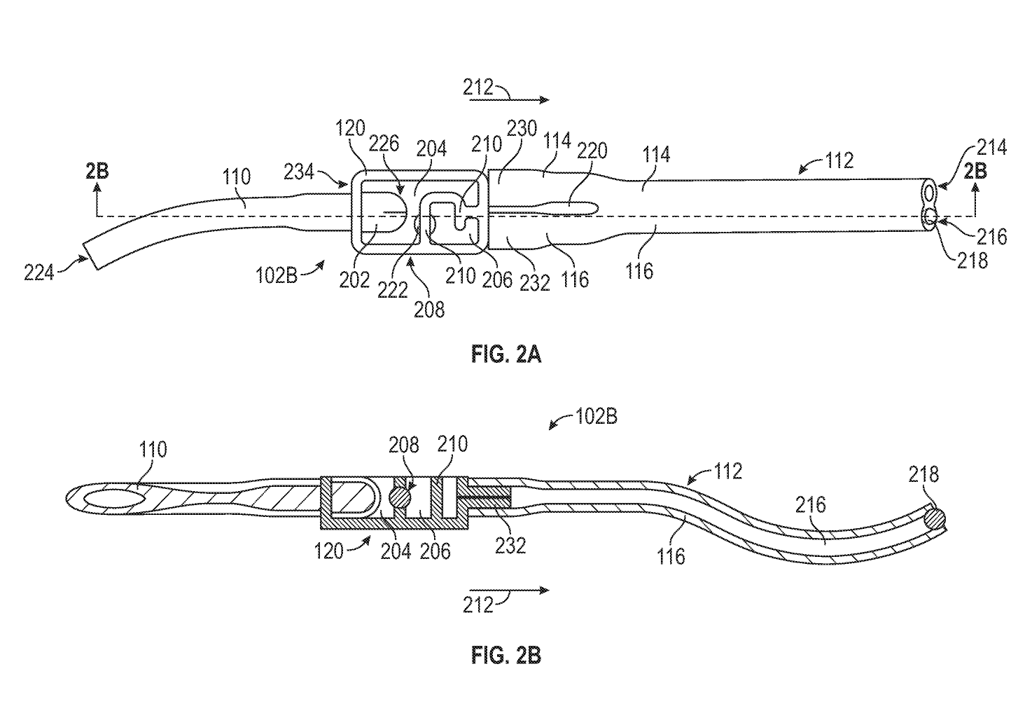

FIG. 2A is a view of a portion of an example bimodal shunt 102B, and FIG. 2B is a cross-section view of the bimodal shunt 102B taken at cut line 2B-2B, in accordance with at least one example of the present disclosure. For ease of illustration, the wall of the chamber body 120 is transparent, such that the inner features of the chamber body 120 can be viewed. In at least one example, the proximal catheter 110 can include a pressure modulation valve 202 to modulate the flow of cerebral spinal fluid into the chamber body 120. In some examples, the pressure modulation valve 202 requires a threshold pressure to initiate flow to the chamber body 120 to prevent the intracranial pressure from dropping too low, as it would in a wide open system. Over-drainage can be dangerous, leading to neurological complications or other issues. While illustrated as a slit valve, the pressure modulation valve 202 can be any passive or actively controlled valve to prevent the intracranial pressure from dropping below a minimum intracranial pressure.

In at least one example, the pressure modulation valve 202 can be separate and proximal to the chamber body 120, such that the chamber body 120 is downstream from the pressure modulation valve 202. In some examples, the distal catheter 112 can be bimodal, including the primary distal catheter 114 and the secondary distal catheter 116. In some examples, the chamber body 120 can include a primary chamber 204 in fluid communication with the primary distal catheter 114 and a secondary chamber 206 in fluid communication with the secondary distal catheter 116. In some examples, the secondary chamber 206 can include a pressure-sensitive valve 208. In some examples, the chamber body 120 includes a primary distal port 230 configured to couple to the primary distal catheter 114, a secondary distal port 232 configured to couple to the secondary distal catheter 116, and a proximal port 234 configured to couple to the proximal catheter 110.

In at least one example, the pressure-sensitive valve 208 can include a ball 222 and one or more wall features 210 configured to restrict movement of the ball. In some examples, pressure in the primary chamber 204 can move the ball 222 into the secondary chamber 206. In at least one example, one or more of the wall features can maintain patency of the secondary distal catheter 116, or otherwise prevent the ball from blocking fluid flow to the secondary distal catheter 116 after the ball has been unseated. In other examples, the pressure-sensitive valve 208 can comprise any valve that blocks flow to the secondary distal catheter 116 until the primary distal catheter 114 fails, for example a fusible link, a slit valve, or the like, and may or may not include one or more wall features 210. In some examples, the pressure-sensitive valve 208 can be a fusible link or ball valve to prevent back pressure, since adding back pressure to that of the pressure modulation valve can lead to persistently elevated intracranial pressure. In some examples, the pressure-sensitive valve 208 can be used to allow for automatic closure of the secondary distal catheter 116 responsive to reopening of the primary distal catheter 114 (e.g., if the occlusion clears in the primary distal catheter 114). In some examples, the pressure-sensitive valve 208 can include a friction fit that can be overcome by a threshold pressure in the primary chamber 204. In at least one example, the pressure-sensitive valve 208 can be configured to remain in a first mode (e.g., closed) blocking flow of fluid from the primary chamber 204 to the secondary chamber 206 until a threshold pressure is reached in the primary chamber 204. In the illustrated example, the proximal catheter 110 can receive and facilitate flow of fluid in a flow direction 212, such that the cerebral spinal fluid flows from a proximal end 224 of the proximal catheter 110 to a distal end 226 of the proximal catheter, into the primary chamber 204 of the chamber body 120, into a proximal end of the primary distal catheter 114, and out a distal end of the primary distal catheter 114 to be drained into the patient's torso. If the primary distal catheter 114 becomes occluded, such that the cerebral spinal fluid is not able to continue its flow in the flow direction 212 out of the primary distal catheter 114, the cerebral spinal fluid will back up and begin to collect in the primary chamber 204, increasing the pressure in the primary chamber 204. In the illustrated example, the increased pressure in the primary chamber 204 can cause the pressure-sensitive valve 208 to transition to a second mode (e.g. open), such that the fluid can flow in the flow direction 212 into the secondary distal catheter 116. In at least one example, the threshold pressure in primary chamber 204 can be indicative of an increased intracranial pressure. For example, in at least one example, the threshold pressure can be indicative of at least 20 cm water intracranial pressure, 15 mmHg intracranial pressure, significantly above normal intracranial pressure, or the like. In at least one example, the threshold pressure in the primary chamber 204 can be about 15 mmHg. In some examples, when the threshold pressure is exceeded, the fluid is permitted to flow from the chamber body 120 to the secondary distal catheter 116. In at least one example, the valve can transition between more than 2 modes. In at least one example, the valve can transition from the second mode to the first mode.

In the illustrated example, each of the primary and secondary distal catheters 114, 116 defines a lumen 214, 216, respectively. In some examples, the primary and secondary distal catheter lumina 214, 216 are distinct, such that the primary distal catheter lumen 214 does not intersect the secondary distal catheter lumen 216. In some examples, the primary and secondary distal catheters 114, 116 can be coupled together along at least a portion of their lengths. In at least one example, the primary and secondary distal catheters 114, 116 can be separated for at least a portion of their lengths, such that the primary and secondary distal catheters 114, 116 can define a gap 220 therebetween.

In some examples, the secondary distal catheter 116 can include a pressure-sensitive valve 218. In the illustrated example, the pressure-sensitive valve 218 includes a ball inserted into the secondary distal catheter lumen 216 via a friction fit. In other examples, the pressure-sensitive valve 218 can comprise any valve that blocks the secondary distal catheter 116 until the primary distal catheter 114 fails, for example a fusible like, a slit valve, or the like. In some examples, the pressure-sensitive valve 218 can block the secondary distal catheter lumen 216 until a threshold pressure is achieved in the secondary distal catheter 116. In at least one example, the threshold pressure for the pressure-sensitive valve 218 in the secondary distal catheter 116 is lower than the threshold pressure for the pressure-sensitive valve 208 in the secondary chamber 206. In some examples, the threshold pressure is high enough that the ball of the pressure-sensitive valve 218 will not fall out of the secondary distal catheter 116 prematurely.

In at least one example, the pressure-sensitive valve 208 and the pressure-sensitive valve 218 together can maintain a pristine secondary distal catheter lumen 216 until the primary distal catheter 114 fails. In the illustrated example, after the threshold pressure has been reached in the primary chamber 204 and cerebral spinal fluid flows into the secondary distal catheter 116 in the fluid direction 212, pressure will build in the secondary distal catheter 116 until a threshold pressure is reached, transitioning the pressure-sensitive valve 218 from a first mode to a second mode, permitting flow in the flow direction 212 out of the secondary distal catheter 116. In at least one example, after both pressure-sensitive valves 208, 218 are transitioned from the first mode to the second mode (e.g., opened), the primary distal catheter 114 is abandoned, and only the secondary distal catheter 116 facilitates drainage of the cerebral spinal fluid. For example, when the primary distal catheter 114 becomes occluded such that it can no longer drain fluid, rather than flushing or replacing the shunt, which can require dangerous and costly surgery, the shunt can rely on the secondary distal catheter 116 without needing the primary distal catheter 114 to convey fluid.

In at least one example, all of the materials of the shunt are biocompatible. In at least one example, the shunt can include polypropylene, silicone, polyurethane (PCU), a combination of these, or the like. In at least one example, the valve body can comprise a rigid material. In at least one example, the catheters can comprise a compliant material. In at least one example, the ball can comprise an elastomeric material. In at least one example, one of the ball and seat (e.g. the wall features) is rigid, while the other is compliant. In at least one example, the pressure-sensitive valve 208 can include an elastomeric ball seated in rigid wall features of the chamber body 120. In at least one example, the pressure-sensitive valve 218 can include a rigid ball seated in the distal end of the elastomeric secondary distal catheter 116.

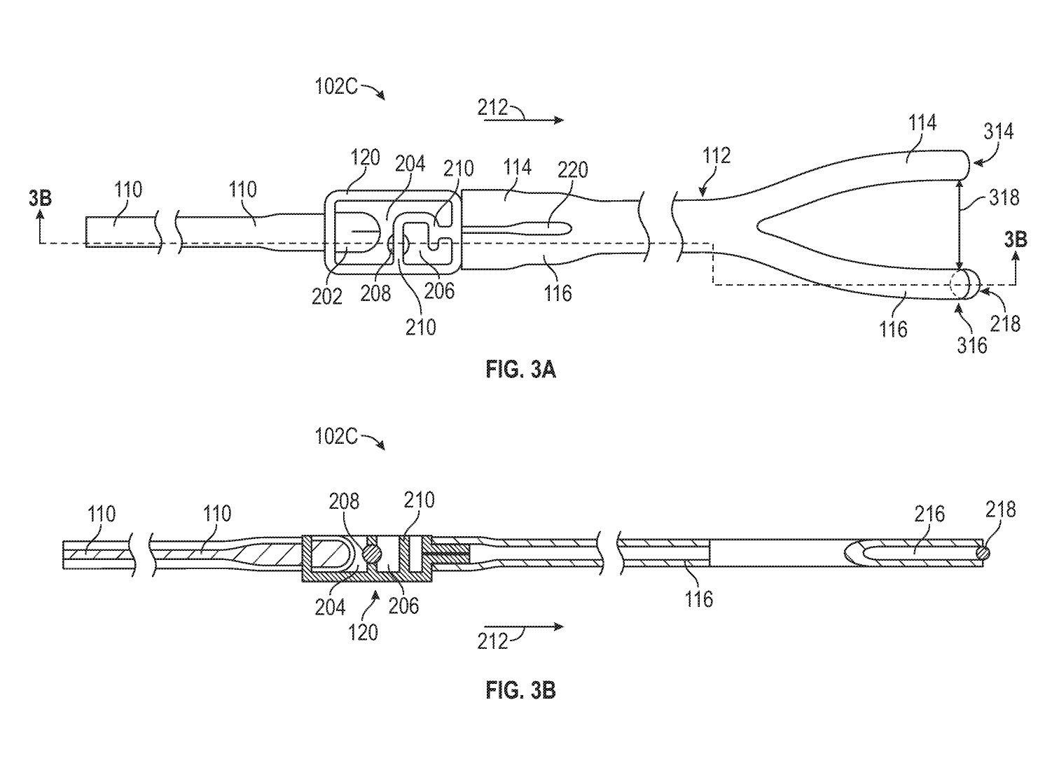

FIG. 3A is a view of a portion of a bimodal shunt 102C, and FIG. 3B is a cross-section view taken at cut line 3B-3B, in accordance with at least one example of the present disclosure. In some examples, a distal end 314 of the primary distal catheter 114 can be splayed apart from a distal end 316 of the secondary distal catheter 116, such that the secondary distal catheter 116 can be positioned a distance 318 away from the primary distal catheter 114. This distance 318 or separation can help extend the life of the bimodal shunt 102C. For example, if a pseudocyst grew over the primary distal catheter 114, there is less chance of the pseudocyst also growing over the secondary distal catheter 116 if the secondary distal catheter 116 is positioned away from the primary distal catheter 114. In different examples, the distal ends 314, 316 of the primary and secondary distal catheters 114, 116 can be splayed any of a range of distances 318. In at least one example, the distal ends 314, 316 can be splayed apart at least a distance of 3 cm. In at least one example, the primary and distal catheters 114, 116 can differ in length, which can facilitate separation of the discharge location of the distal ends.

In at least one example, the bimodal shunt 102C can be implanted with the distal ends 314, 316 of the primary and distal catheters 114, 116 together to facilitate ease of insertion. For example, the distal ends 314, 316 can be separated in a resting state, but a surgeon can position the distal ends 315, 316 side-by-side, such that the distal ends 314, 316 can be tunneled beneath a patient's skin. In at least one example, the ends 314, 416 can splay apart a distance 318 once the distal catheters 114, 116 are implanted in a patient's body. In at least one example, the distal ends 314, 316 can be together as they are tunneled beneath the skin to the peritoneum, and then splay apart.

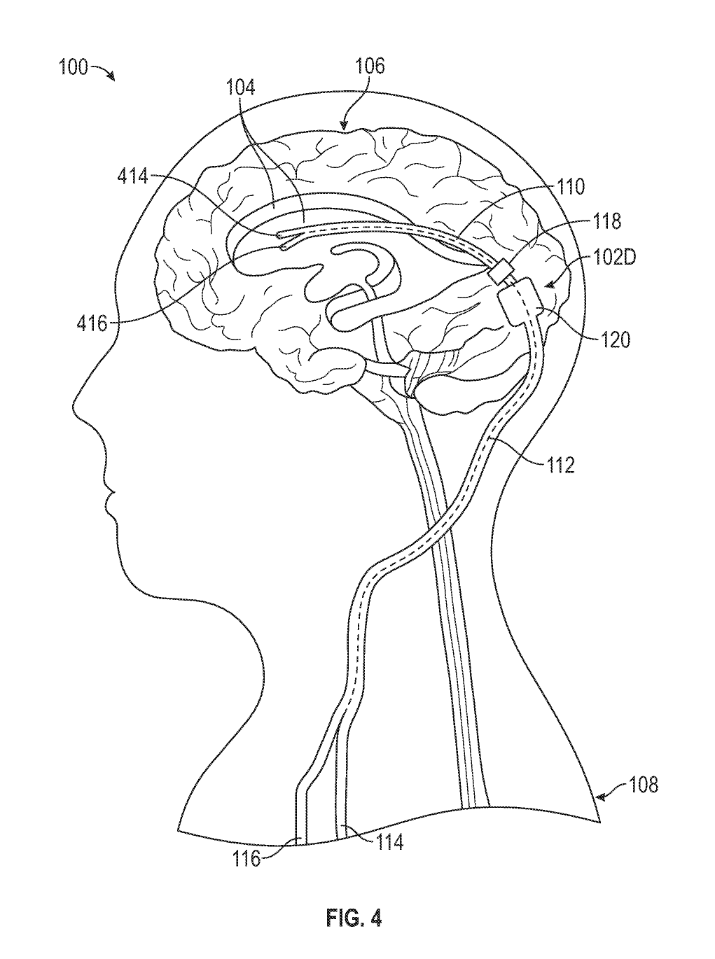

FIG. 4 is a diagram of a patient 100 having an implanted bimodal cerebral spinal fluid shunt 102D, in accordance with at least one example of the present disclosure. As illustrated, in some examples, the proximal catheter 110 can include a primary proximal catheter 414 and a secondary proximal catheter 416. In at least one example, the primary and secondary proximal catheters are ventricular catheters. In the illustrated example, the bimodal shunt 102D includes both proximal and distal redundancy, however, in some examples, the proximal catheter 110 can have redundancy while the distal catheter 112 does not. In such an example, the chamber body 120 can be omitted, such that the pressure modulation valve 118 can be directly coupled to the distal catheter 112. In at least one example, the bimodal shunt 102D can include proximal redundancy and distal redundancy with the chamber body 120 omitted. In at least one example, the primary proximal catheter 414 is in fluid communication with the primary distal catheter 114, and the secondary proximal catheter 416 is in fluid communication with the secondary distal catheter 116, while the respective lumina of the primary catheters 414, 114 are distinct from and not in fluid communication with respective lumina of the secondary catheters 416, 116. In such an example, the primary and secondary catheters are connected along at least a portion of their length. In at least one example, the bimodal shunt 102D can include two pressure modulation valves 118, one for the primary catheters 414, 114 and one for the secondary catheters 416, 416. In some examples, the secondary proximal catheter 416 can remain pristine until the primary proximal catheter 414 fails, at which point the secondary proximal catheter 416 can become active and the primary proximal catheter 414 can be abandoned.

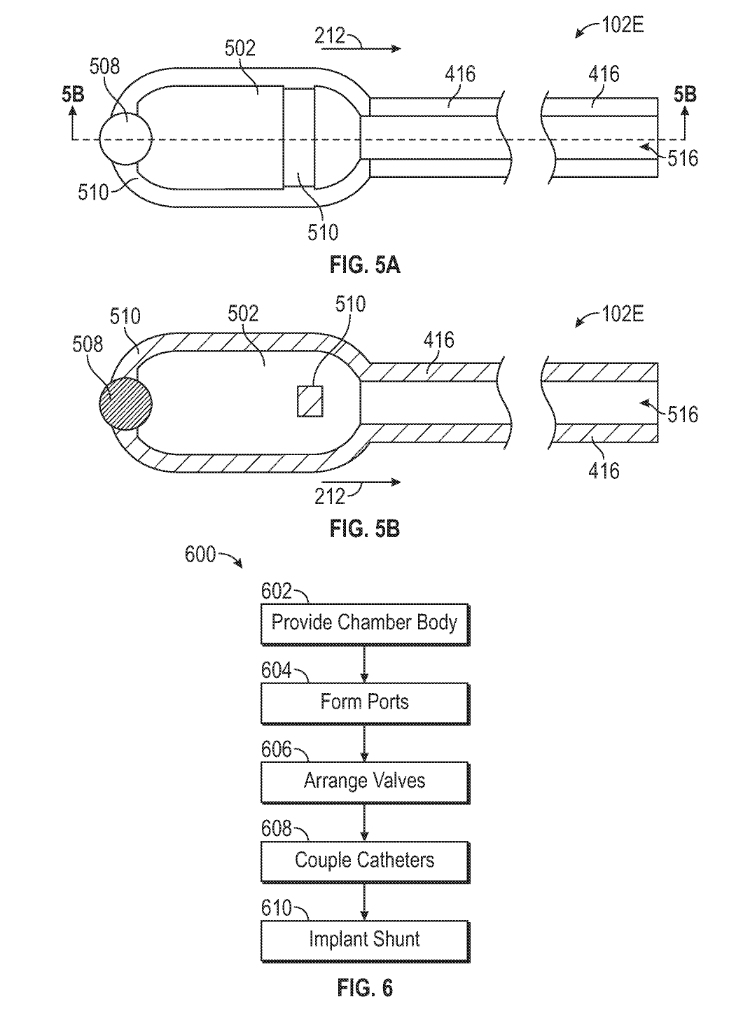

FIG. 5A is a view of a portion of a bimodal shunt 102E (shown with transparency so as to reveal the inner features) and FIG. 5B is a cross-section view of the portion of the bimodal shunt 102E taken at cut line 5B-5B, in accordance with at least one example of the present disclosure. The illustrated example shows the secondary proximal catheter 416 including a proximal chamber 502 and a pressure-sensitive valve 508. In some examples, the proximal chamber 502 can be positioned at a proximal end of the secondary proximal catheter 416. In some examples, the pressure-sensitive valve 508 can be positioned at a proximal end of the secondary proximal catheter 416 and the proximal chamber 502 can be omitted. In at least one example, the pressure-sensitive valve 508 can prevent flow in the flow direction 212 to the secondary proximal catheter 416 until a threshold intracranial pressure is reached, at which point the pressure-sensitive valve 508 automatically transitions from the first mode to the second mode, permitting flow to the secondary proximal catheter 416. In some examples, the threshold intracranial pressure is any intracranial pressure above a normal intracranial pressure. In at least one example, the threshold intracranial pressure can be 15 cm water. In at least one example, the threshold intracranial pressure can be 11 mmHg. In at least one example, the threshold intracranial pressure can be 20 cm water, 15 mmHg, significantly above normal intracranial pressure, or the like.

In the illustrated example, the pressure-sensitive valve 508 includes a ball positioned with a friction fit that can be overcome by a threshold intracranial pressure. In some examples, the proximal chamber 502 or the secondary proximal catheter 416 can include one or more wall features 510 to restrict the movement of the ball 508. For example, the wall feature 510 can prevent the ball 508 from blocking the lumen 516 of the secondary proximal catheter 416 after the ball 508 has been unseated due to failure of the primary proximal catheter 414. In other examples, the pressure-sensitive valve 508 can comprise any valve that blocks flow to the secondary proximal catheter 416 until the primary proximal catheter 414 fails, for example, a slit valve, a fusible link, or the like. In at least one example, the lumen 516 of the secondary proximal catheter 416 is kept in pristine condition until the primary proximal catheter 414 fails due to occlusion or otherwise, at which point the intracranial fluid will begin to build up in the ventricles until a threshold pressure is reached, transitioning the pressure-sensitive valve 508 from a first mode to a second mode, permitting flow of the cerebral spinal fluid in the flow direction 212 into the secondary proximal catheter 416. In an example including distal redundancy, the cerebral spinal fluid would flow in the flow direction 212 from the secondary proximal catheter 416 to the chamber body 120 and into the primary distal catheter 114, while the secondary distal catheter 116 (see FIG. 4) remains pristine until failure of the primary distal catheter 114.

FIG. 6 is a flow chart illustrating a method 600, in accordance with at least one example of the present disclosure. The method 600 will be described with reference to FIGS. 1-5B, the features of which may be combined in any of a variety of configurations. At block 602, a chamber body 120 can be provided. In at least one example, the chamber body 120 is an implantable chamber body.

At block 604, one or more ports can be formed on the chamber body 120. In at least one example, a proximal port 234 can be formed on the chamber body 120. In at least one example, a primary distal port 230 and a secondary distal port 232 can be formed on the chamber body.

At block 606, one or more valves can be arranged. In at least one example, a pressure-sensitive valve 208 can be arranged in the chamber body 120. In some examples, the pressure-sensitive valve 208 can include a first mode and a second mode. In at least one example, the primary distal catheter port 230 is in fluid communication with the proximal catheter port 234 and the secondary distal catheter port 232 is not in fluid communication with the proximal catheter port 234 when the pressure-sensitive valve 208 is in the first mode. In at least one example, secondary distal catheter port 232 is in fluid communication with the proximal catheter port 234 when the pressure-sensitive valve 208 is in the second mode.

In at least one example, arranging the pressure-sensitive valve 208 in the chamber body 120 includes configuring the pressure-sensitive valve 208 to transition from the first mode to the second mode responsive to a threshold fluid pressure in the chamber body 120. In at least one example, arranging the pressure-sensitive valve 208 in the chamber body 120 includes providing one or more wall features 210 to maintain patency of the secondary distal catheter port 232 when the pressure-sensitive valve 208 is in the second mode. In at least one example, arranging the pressure-sensitive valve 208 in the chamber body 120 includes configuring the pressure-sensitive valve 208 to irrevocably remain in the second mode.

In some examples, a distal valve 218 can be arranged in a secondary distal catheter 116. In at least one example, the distal valve 218 can be formed at a distal end 216 of the secondary distal catheter 116. In some examples, the distal valve 218 can include a pressure-sensitive valve including at least a first mode and a second mode. In at least one example, the first mode restricts fluid flow through the distal end 316 of the secondary distal catheter 116 and the second mode permits fluid flow through the distal end 316 of the secondary distal catheter 116. In some examples, the pressure-sensitive valve 218 is configured to transition from the first mode to the second mode responsive to a threshold fluid pressure in the secondary distal catheter 116. In at least one example, the distal valve 218 plugs the distal end 316 of the secondary distal catheter 116 in the first mode and is configured to eject from the distal end 316 in the second mode.

At block 608, one or more catheters can be coupled to one or more ports of the chamber body 120. In at least one example, a proximal catheter 110 can be coupled to the proximal port 234. In some examples, a primary distal catheter 114 can be coupled to the primary distal port 230 and a secondary distal catheter 116 can be coupled to the secondary distal port 232. In at least one example, one or more catheters can be in fluid communication with the chamber body 120 via the one or more ports.

At block 610, the chamber body, one or more ports, and one or more catheters can form an implantable shunt that can be implanted into a patient's anatomy. In at least one example, the shunt can be a cerebral spinal fluid shunt configured to drain cerebral spinal fluid.

As described in FIGS. 1-6, in some examples, a bimodal shunt can passively detect failure of a primary catheter and automatically redirects flow to a secondary catheter to increase the useful lifespan of the shunt.

In the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus the following claims are hereby incorporated into the Detailed Description, with each claim standing on its own as a separate example.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific examples. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific examples. However, the benefits, advantages, solutions to problems, and any feature(s) that may cause any benefit, advantage, or solution to occur or become more pronounced are not to be construed as a critical, required, or essential feature of any or all the claims. Moreover, the particular examples disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular examples disclosed above may be altered or modified and all such variations are considered within the scope of the disclosed subject matter. Accordingly, the protection sought herein is as set forth in the claims below.

What is claimed is:

1. A shunt comprising:
a primary catheter configured to receive fluid;
a secondary catheter configured to receive cerebral spinal fluid; and
a first pressure-sensitive valve positioned to prevent flow of cerebral spinal fluid to the secondary catheter in a first mode;
wherein the first pressure-sensitive valve is configured to transition to a second mode responsive to occlusion of the primary catheter, permitting flow of cerebral spinal fluid to the secondary catheter.

2. The shunt of claim 1, wherein the primary and secondary catheters are ventricular catheters.

3. The shunt of claim 2, wherein:
the first pressure-sensitive valve is positioned at a proximal end of the secondary catheter; and
the first pressure-sensitive valve is configured to transition to the second mode responsive to a threshold intracranial pressure.

4. The shunt of claim 1, wherein the first pressure-sensitive valve comprises a ball and at least one wall feature, the at least one wall feature configured to restrict movement of the ball.

5. The shunt of claim 1, further comprising:
a second pressure-sensitive valve positioned at a distal end of the secondary catheter to block flow through the distal end of the secondary catheter in a first mode and configured to transition to a second mode responsive to a threshold pressure in the secondary catheter.

6. The shunt of claim 1, further comprising:
a chamber coupled to a proximal end of the secondary catheter, wherein the first pressure-sensitive valve is configured to transition to the second mode responsive to a threshold pressure in the chamber.

7. A shunt comprising:
a chamber body configured to receive fluid;
a primary distal catheter coupled at a proximal end to the chamber body and configured to receive fluid from the chamber body;
a secondary distal catheter coupled at a proximal end to the chamber body and configured to receive fluid from the chamber body; and
a first pressure-sensitive valve configured to prevent flow to the secondary distal catheter from the chamber body in a first mode until the primary distal catheter is occluded such that a threshold pressure is reached in the chamber body to transition the first pressure-sensitive valve to a second mode.

8. The shunt of claim 7, wherein:
the chamber body comprises a primary chamber and a secondary chamber; and
the first pressure-sensitive valve is positioned to prevent flow to the secondary chamber until a threshold pressure is reached in the primary chamber.

9. The shunt of claim 7, further comprising:
a pressure modulation valve configured to modulate the flow of fluid to the chamber body.

10. The shunt of claim 7, further comprising:
a second pressure-sensitive valve positioned at a distal end of the secondary distal catheter, the second pressure-sensitive valve configured to block flow through the distal end of the secondary distal catheter in a first mode and to transition to a second mode responsive to a threshold pressure in the secondary distal catheter.

11. The shunt of claim 7, wherein the primary and secondary distal catheters are coupled together along a portion of their lengths.

12. The shunt of claim 7, wherein the first pressure-sensitive valve comprises a fusible link.

13. The shunt of claim 7, wherein a distal end of the secondary distal catheter is configured to be positioned away from a distal end of the primary distal catheter, such that the distal ends of the primary and secondary distal catheters can be splayed apart.

14. The shunt of claim 13, wherein the distal ends of the primary and secondary distal catheters are configured to be positioned together.

15. A shunt comprising:
a proximal catheter configured to receive fluid for draining;
a chamber body configured to receive fluid from the proximal catheter, the chamber body including a primary chamber and a secondary chamber; and
a first pressure-sensitive valve positioned in the chamber body to prevent flow of fluid to the secondary chamber in a first mode;
wherein the first pressure-sensitive valve is configured to transition to a second mode responsive to a threshold pressure in the primary chamber.

16. The shunt of claim 15, further comprising:
a primary distal catheter in fluid communication with the primary chamber; and
a secondary distal catheter in fluid communication with the secondary chamber, wherein the first pressure-sensitive valve is configured to permit flow to the secondary distal catheter responsive to failure of the primary distal catheter.

17. The shunt of claim 16, further comprising:
a second pressure-sensitive valve positioned at a distal end of the secondary distal catheter to block flow through the distal end of the secondary distal catheter in a first mode and to transition to a second mode responsive to a threshold pressure in the secondary distal catheter.

18. The shunt of claim 17, wherein the second pressure-sensitive valve comprises a friction fit ball.

19. The shunt of claim 15, wherein the first pressure-sensitive valve comprises a ball and at least one wall feature configured to restrict movement of the ball.

20. The shunt of claim 15, further comprising:
a pressure modulation valve coupled to a distal end of the proximal catheter and configured to receive fluid from the proximal catheter.

21. A method comprising:
providing an implantable chamber body;
forming a proximal catheter port on the chamber body;
arranging a pressure-sensitive valve in the chamber body, the valve including a first mode and a second mode; and
forming a primary distal catheter port and a secondary distal catheter port on the chamber body;
wherein the pressure-sensitive valve permits the primary distal catheter port to be in fluid communication with the proximal catheter port and blocks the secondary distal catheter port from being in fluid communication with the proximal catheter port in the first mode;
wherein the pressure-sensitive valve permits the secondary distal catheter port to be in fluid communication with the proximal catheter port when the pressure-sensitive valve is in the second mode.

22. The method of claim 21, wherein arranging the pressure-sensitive valve in the chamber body includes affixing a ball in a seat.

23. The method of claim 21, wherein arranging the pressure-sensitive valve in the chamber body includes providing one or more wall features to maintain patency of the secondary distal catheter port when the pressure-sensitive valve is in the second mode.

24. The method of claim 21, wherein arranging the pressure-sensitive valve in the chamber body includes configuring the pressure-sensitive valve to transition from the first mode to the second mode responsive to a threshold fluid pressure in the chamber body.

25. The method of claim 21, wherein arranging the pressure-sensitive valve in the chamber body includes configuring the pressure-sensitive valve to irrevocably remain in the second mode.

26. The method of claim 21, further including:
coupling a proximal catheter to the proximal catheter port;
coupling a primary distal catheter to the primary distal catheter port; and
coupling a secondary distal catheter to the secondary distal catheter port.

27. The method of claim 26, further comprising:
arranging a distal valve at a distal end of the secondary distal catheter.

28. The method of claim 27, wherein the distal valve comprises a pressure-sensitive valve including a first mode restricting fluid flow through the distal end of the secondary distal catheter and a second mode permitting fluid flow through the distal end of the secondary distal catheter.

29. The method of claim 28, wherein the pressure-sensitive valve is configured to transition from the first mode to the second mode responsive to a threshold fluid pressure in the secondary distal catheter.

30. The method of claim 29, wherein the distal valve plugs the distal end of the secondary distal catheter in the first mode and is configured to eject from the distal end in the second mode.
